(19) United States
(12) Patent Application Publication
(10) Pub. No.: US 2011/0313187 A1
(43) Pub. Date: Dec. 22, 2011

(54) METHOD FOR IMPROVING THE CATALYTIC ACTIVITY OF MONOLITHIC CATALYSTS

(76) Inventors: Christof Wilhelm Wigbers, Mannheim (DE); Jochen Steiner, Bensheim (DE); Martin Ernst, Heidelberg (DE); Bram Willem Hoffer, Fanwood, NJ (US); Ekkehard Schwab, Neustadt (DE); Johann-Peter Melder, Bohl-Iggelheim (DE)

(21) Appl. No.: 13/148,586

(22) PCT Filed: Feb. 1, 2010
(86) PCT No.: PCT/EP2010/051143

§ 371 Date: Aug. 9, 2011

(30) Foreign Application Priority Data
Feb. 9, 2009 (EP) 09152406.6

(51) Int. Cl. C07C 209/48; C07C 253/30

(52) U.S. Cl. 558/459; 564/490; 564/491; 564/448; 564/492

(57) ABSTRACT

The present invention relates to a process for improving the catalytic properties of a catalyst comprising one or more elements selected from the group consisting of cobalt, nickel and copper, said catalyst being present in the form of a structured monolith, by contacting the catalyst with one or more basic compounds selected from the group of the alkali metals, alkaline earth metals and rare earth metals. The invention further relates to a process for hydrogenating compounds which comprise at least one unsaturated carbon-carbon, carbon-nitrogen or carbon-oxygen bond in the presence of a catalyst comprising one or more elements selected from the group consisting of cobalt, nickel and copper, said catalyst being present in the form of a structured monolith, by contacting the catalyst with one or more basic compounds selected from the group of the alkali metals, alkaline earth metals and rare earth metals. The present invention also relates to the use of a basic compound selected from the group of the alkali metals, alkaline earth metals and rare earth metals for improving the catalytic properties of a catalyst comprising cobalt and/or copper and/or nickel, said catalyst being present in the form of a structured monolith.

The present invention relates to a process for improving the catalytic properties of a catalyst comprising one or more elements selected from the group consisting of cobalt, nickel and copper, said catalyst being present in the form of a structured monolith, by contacting the catalyst with one or more basic compounds selected from the group of the alkali metals, alkaline earth metals and rare earth metals. The invention further relates to a process for hydrogenating compounds which comprise at least one unsaturated carbon-carbon, carbon-nitrogen or carbon-oxygen bond in the presence of a catalyst comprising one or more elements selected from the group consisting of cobalt, nickel and copper, said catalyst being present in the form of a structured monolith, by contacting the catalyst with one or more basic compounds selected from the group of the alkali metals, alkaline earth metals and rare earth metals. The present invention also relates to the use of a basic compound selected from the group of the alkali metals, alkaline earth metals and rare earth metals for improving the catalytic properties of a catalyst comprising cobalt and/or copper and/or nickel, said catalyst being present in the form of a structured monolith.

The preparation of amines by hydrogenating nitriles is effected generally in the presence of catalysts which comprise the elements Cu, Ni and Co.

In nitrile hydrogenation, a frequent side reaction which occurs is the formation of secondary amines.

The occurrence of this side reaction can be reduced when the hydrogenation is performed in the presence of ammonia (see Ullman's Encyclopedia of Industrial Chemistry, 6th edition, volume 2, p. 385). For an effective reduction in the formation of secondary amines, however, relatively large amounts of ammonia are required. The handling of ammonia is additionally technically complex, since it has to be stored, handled and reacted under high pressure.

U.S. Pat. No. 2,449,036 discloses that the formation of secondary amines in the case of use of activated nickel or cobalt sponge catalysts can be effectively suppressed even in the absence of ammonia when the hydrogenation is performed in the presence of a strong base, such as alkali metal or alkaline earth metal hydroxides.

WO 92/21650 describes the use of further bases, such as alkali metal alkoxides and alkali metal carbonates, in hydrogenation with Raney catalysts.

EP-A1-913388 teaches that good selectivities and yields of primary amines are achieved in nitrile hydrogenation when working in the presence of water and a suspended Raney cobalt catalyst which has been treated with catalytic amounts of LiOH.

In order to minimize the leaching of metals, for example aluminum in the case of skeletal catalysts or alkaline promoters such as lithium, out of the catalyst, WO 2007/104663 described mixed oxide catalysts, especially LiCoO₂, in which the alkali metal atoms are incorporated in the crystal lattice.

In the above-described processes, the catalysts are generally used in the form of unsupported catalysts, i.e. the catalyst consists almost completely of catalytically active material. In the prior art cited, the hydrogenation is generally performed in suspension. This means that the catalysts, after the reaction has ended, have to be removed from the reaction mixture by filtration.

WO 2007/028411 gives an overview of the preparation of supported Raney-type catalysts. It is stated here that these catalysts have several disadvantages, including their low mechanical stability, their comparatively low activity and their complicated preparation. Supported Raney catalysts with improved properties are said by the disclosure of WO 2007/028411 to be achieved by coating support materials with nickel/aluminum, cobalt/aluminum or copper/aluminum alloys. The catalysts thus prepared are activated by leaching out all or a portion of the aluminum with a base.

A further approach to the preparation of supported catalysts which are said to be suitable for nitrile hydrogenation is described in WO 2006/079850. These catalysts are obtained by applying metals to a structured monolith, the application being effected by impregnating the monolith with a solution in which the metal is present as an ammine complex. According to the disclosure, the catalysts thus prepared are suitable for a series of chemical reactions, one of which cited is the hydrogenation of nitriles. With regard to the hydrogenation of nitriles, WO 2006/079850, however, does not constitute a performable disclosure, since it does not give details, instructions or experiments for this reaction type.

By means of this invention, the catalytic properties of catalysts present in the form of a structured monolith should be improved.

In particular, the formation of undesired by-products, particularly the formation of secondary amines from nitriles, should be reduced in order to obtain the target products in a high yield and selectivity. Furthermore, the service life of the catalysts should be improved and the losses in activity and selectivity with increasing operating time should be reduced. It was a further aim to reestablish the catalytic properties of spent catalysts.

Accordingly, a process has been found for improving the catalytic properties of a catalyst comprising one or more elements selected from the group consisting of cobalt, nickel and copper, said catalyst being present in the form of a structured monolith, which comprises contacting the catalyst with one or more basic compounds selected from the group of the alkali metals, alkaline earth metals and rare earth metals.

The catalyst used in the process according to the invention comprises one or more elements selected from the group consisting of cobalt, nickel and copper. The catalyst preferably comprises cobalt or nickel and, in a preferred embodiment, the catalyst comprises cobalt.

In a particularly preferred embodiment, the inventive catalysts further comprise one or more elements selected from the group of the alkali metals, alkaline earth metals and rare earth metals.

In the context of this invention, it has been found that the presence of one or more elements of the alkali metals, alkaline earth metals and rare earth metals brings an additional improvement in the catalytic and in the mechanical properties.

Preferred elements of the group of the alkali metals are Li, Na, K, Rb and Cs, particular preference being given to Li, Na, K and Cs, especially Li, Na and K.

Preferred elements of the group of the alkaline earth metals are Be, Mg, Ca, Sr and barium, particular preference being given to Mg and Ca.

Preferred elements of the group of the rare earths are Sc, Y, La, Ce, Pr, Nd, Pm, Sm, Eu, Gd, Tb, Dy, Ho, Er, Tm, Yb and Lu, particular preference being given to Sc, Y, La and Ce.

When the catalyst comprises Ni, the catalyst comprises, in a particularly preferred embodiment, Na as the alkali metal. Further preferred combinations are Ni and Li, Ni and K, and Ni and Cs.

When the catalyst comprises Co, the catalyst comprises, in a particularly preferred embodiment, Li as the alkali metal. Further preferred combinations are Co and Na, Co and K and Co and Cs.

The catalyst may optionally comprise one or more doping elements.

The doping elements are preferably selected from the elements of transition groups 3 to 8 and main groups 3, 4 and 5 of the Periodic Table of the Elements.

Preferred doping elements are Fe, Ni, Cr, Mo, Mn, P, Ti, Nb, V, Cu, Ag, Pd, Pt, Rh, Ir, Ru and Au.

The molar ratio of Cu, Co and Ni atoms to atoms of the elements of the alkali metals, alkaline earth metals and rare earth metals in the catalyst is preferably 0.1:1 to 10 000:1, preferably 0.5:1 to 1000:1 and more preferably 0.5:1 to 500:1.

In a very particularly preferred embodiment, the molar ratio of Cu, Co and Ni atoms to atoms of the elements of the alkali metals, alkaline earth metals and rare earth metals in the catalyst is less than 300:1, preferably less than 100:1, especially preferably less than 50:1 and most preferably less than 25:1.

The molar ratio of Co, Cu and Ni atoms to atoms of the doping elements is preferably 10:1 to 100 000:1, preferably 20:1 to 10 000:1 and more preferably 50:1 to 1000:1.

The term “catalytically active components” is used hereinafter for the elements Cu, Co, Ni, the elements of the alkali metals, alkaline earth metals and rare earth metals, and the doping elements mentioned, i.e. the elements of transition groups 3 to 8 and of main groups 3, 4 and 5 of the Periodic Table of the Elements.

The molar ratio of the atoms of the components of the active material relative to one another can be measured by means of known methods of elemental analysis, for example of atomic absorption spectrometry (AAS), of atomic emission spectrometry (AES), of X-ray fluorescence analysis (RFA) or of ICP-OES (Inductively Coupled Plasma-Optical Emission Spectrometry). The molar ratio of the atoms of the components of the active material relative to one another can, however, also be determined arithmetically, for example by determining the starting weights of the compounds used, which comprise the components of the active material, and determining the proportions of the atoms of the components of the active material on the basis of the known stoichiometry of the compounds used, such that the atomic ratio can be calculated from the starting weights and the stoichiometric formula of the compound used. Of course, the stoichiometric formula of the compounds used can also be determined experimentally, for example by one or more of the above-mentioned methods.

The inventive catalyst is present in the form of a structured monolith. The term “structured monolith” is understood to mean shaped bodies which have been shaped to a body which comprises a multitude of penetrating (or connected) channels through which the reactants and products are transported by flow/convection.

In the context of this invention, accordingly, the term “structured monolith” is understood to mean not just the “conventional” shaped bodies with parallel channels not connected radially to one another, but also shaped bodies in the form of foams, sponges or the like with three-dimensional connections within the shaped body. The term “monolith” also includes shaped bodies with crossflow channels.

The number of channels in the structured monolith per square inch, which is also referred to as the “cell density” or “cells per square inch (cpsi)”, is preferably 5 to 2000 cpsi, more preferably 25 to 1000 cpsi, especially preferably 250 to 900 cpsi and most preferably 400 to 900 cpsi.

The inventive catalysts can be converted to the form of a structured monolith by mixing the catalytically active components or the compounds of the catalytically active components with a catalyst framework material, and shaping them to a structured monolith. The preparation can be effected, for example, analogously to the preparation method described in EP-A2-1147813, by mixing the catalytically active components with the catalyst framework material and optionally further additives, such as binders and deforming assistants, and extruding them to honeycombs with appropriately shaped extrusion dies.

The inventive catalysts are preferably prepared by applying the catalytically active components or the compounds of the catalytically active components to a catalyst framework material, said catalyst framework material already being present in the form of a structured monolith.

In the context of the present invention, catalyst framework material which is present in the form of a structured monolith is referred to as a monolithic catalyst support. Methods of preparing monolithic catalyst supports are known and are described in detail in the publication by Niijhuis et al., Catalysis Reviews 43 (4) (2001), pages 345 to 380, whose contents are incorporated by reference.

As the catalyst framework material, structured monoliths generally comprise ceramic, metals or carbon.

Preferred catalyst framework materials are ceramic materials such as aluminum oxides, especially gamma- or delta-aluminum oxides, alpha-aluminum oxides, silicon dioxide, kieselguhr, titanium dioxide, zirconium dioxide, cerium dioxide, magnesium oxide, and mixtures thereof.

Especially preferred catalyst framework materials are ceramic materials, such as kaolinite and mullite, which are oxide mixtures of SiO₂ and Al₂O₃ in a ratio of approx. 2:3, and also beryllium oxide, silicon carbide, boron nitride or boron carbide.

In a particularly preferred embodiment, the catalyst framework material is cordierite. Cordierite materials and variants based thereon are magnesium aluminum silicates which form directly when soapstone or talc is sintered with additions of clay, kaolin, chamotte, corundum and mullite. The simplified approximation and composition of pure ceramic cordierite is approx. 14% MgO, 35% Al2O3 and 51% SiO2 (source: www.keramikverband.de).

The structured monoliths or monolithic catalyst supports may be of any desired size. The dimensions of the monolithic catalysts are preferably between 1 cm and 10 m, preferably between 10 cm and 5 m and most preferably between 20 cm and 100 cm. The structured monoliths may also have a modular structure formed from individual monoliths in which small monolithic base structures are combined (e.g. adhesive-bonded) to form larger units.

Monolithic catalyst supports are, for example, also commercially available, for example under the Corning Celcor® brand from Corning, and under the HoneyCeram® brand from NGK Insulators Ltd.

In a preferred embodiment, the catalytically active components are applied to a monolithic catalyst support.

The catalytically active components can be applied to the monolithic catalyst support, for example, by impregnation or coating.

The impregnation (also “saturation”) of the monolithic catalyst support can be effected by the customary processes, for example by applying a soluble compound of the catalytically active components in one or more impregnation stages.

Useful soluble compounds of the catalytically active components generally include soluble metal salts, such as the hydroxides, sulfates, carbonates, oxalates, nitrates, acetates or chlorides of the catalytically active components. The impregnation can also be effected with other suitable soluble compounds of the corresponding elements. The elements Cu, Co and/or Ni are preferably used in the form of their soluble carbonates, chlorides or nitrates. However, it is also possible to use soluble ammine complexes of Cu, Ni or Co, as described, for example, in WO 2006/079850.

The elements of the alkali metals, alkaline earth metals and rare earth metals are preferably used in the form of their soluble hydroxides, preferably LiOH, KOH, NaOH, CsOH, Ca(OH)₂ or Mg(OH)₂.

The impregnation is effected typically in a liquid, in which the soluble compounds of the catalytically active elements are dissolved.

The liquids used are preferably water, nitriles, amines, ethers such as tetrahydrofuran or dioxane, amides such as N,N-dimethylformamide or N,N-dimethylacetamide. Particular preference is given to using water as the liquid.

When nitriles are used as the liquid, preference is given to using the nitrile which is to be hydrogenated later with the inventive catalyst. The amines used as liquids are preferably those which form as the product in a subsequent hydrogenation.

The concentration of the soluble compounds of the catalytically active components in the liquid is generally 0.1 to 50% by weight, preferably 1 to 30% by weight and more preferably 5 to 25% by weight, based in each case on the mass of the liquid used. In particular, the concentration of the soluble compounds of the alkali metals, alkaline earth metals and rare earth metals is 0.1 to 25% by weight, preferably 0.5 to 20% by weight, especially preferably 1 to 15% by weight and most preferably 5 to 10% by weight, based in each case on the mass of the liquid used.

The concentration of the soluble compounds of Cu, Ni and Co is 1 to 50% by weight, preferably 5 to 25% by weight and more preferably 10 to 20% by weight, based in each case on the mass of the liquid used.

The impregnation is effected preferably by immersing the monolithic catalyst support into the liquid which comprises the dissolved catalytically active components (impregnation solution).

In a particularly preferred embodiment, during the immersion, the impregnation solution is sucked in through the channels of the monolithic catalyst support, such that the impregnation solution can penetrate very substantially fully into the channels of the monolith. The impregnation solution can be sucked in, for example, by generating a reduced pressure at one end of the monolithic catalyst support and immersing the other end of the monolithic catalyst support into the impregnation solution, which sucks in the impregnation solution.

The impregnation can also be effected by the so-called “incipient wetness method”, in which the monolithic catalyst support, according to its absorption capacity, is moistened up to a maximum of saturation with the impregnation solution. The impregnation can, however, also be effected in supernatant solution. Thereafter, the impregnated monolithic catalyst support is generally removed from the impregnation solution.

The impregnation solution can be removed, for example, by decanting off, dripping off, filtration or filtering off. The impregnation solution is preferably removed by generating an elevated pressure at one end of the monolithic catalyst support and forcing the excess impregnation solution out of the channels. The elevated pressure can be generated, for example, by blowing compressed air into the channels.

After the removal of the impregnation solution, the impregnated monolithic catalyst support is preferably dried and calcined.

The drying is effected typically at temperatures of 80 to 200° C., preferably 100 to 150° C. The calcination is performed generally at temperatures of 300 to 800° C., preferably 400 to 600° C., more preferably 450 to 550° C.

In a preferred embodiment, the impregnation is effected in one or more stages. In multistage impregnation processes, it is appropriate to dry and optionally to calcine between individual impregnation steps. Multistage impregnation should be employed advantageously when the monolithic catalyst support is to be contacted with metal salts in a relatively large amount.

In a very particularly preferred embodiment, in a one-stage or multistage impregnation, in the last impregnation stage, one or more elements selected from the group of the alkali metals, alkaline earth metals and rare earth metals are applied to the monolithic catalyst support by impregnation.

In order that the proportion of elements of the alkali metals, alkaline earth metals and rare earth metals on the monolithic catalyst is at a maximum, it is advantageous when the catalyst, after the application of the elements of the alkali metals, alkaline earth metals and rare earth metals, is not washed or treated in a similar manner which leads to reduction of the content of these elements. The monolithic catalyst supports impregnated with alkali metals, alkaline earth metals and rare earth metals are preferably dried and calcined directly after the impregnation, as described above.

To apply a plurality of components to the monolithic catalyst support, the impregnation can be effected, for example, simultaneously with one or more soluble compounds of the catalytically active components together or in any desired sequence of the individual soluble compounds of the catalytically active components in succession.

In a very particularly preferred embodiment, the catalytically active components are applied by coating.

The coating process generally involves contacting the monolithic catalyst support together with a suspension which comprises one or more insoluble or sparingly soluble compounds of the catalytically active components. In the context of the present invention, gels which comprise the catalytically active components are also included among the sparingly soluble or insoluble compounds. However, the suspension may also additionally comprise one or more soluble compounds of the catalytically active components.

The liquid used, in which the insoluble or sparingly soluble compounds of the catalytically active components or gels thereof are suspended together with the monolithic catalyst support, is preferably water, nitriles, amines, ethers such as tetrahydrofuran or dioxane, amides such as N,N-dimethylformamide or N,N-dimethylacetamide. Particular preference is given to using water as the liquid.

When nitriles are used as the liquid, preference is given to using the nitrile which is to be hydrogenated later with the inventive catalyst. The amines used as liquids are preferably those amines which form as the product in a subsequent hydrogenation. The insoluble or sparingly soluble compounds of the catalytically active components are preferably oxygen-containing compounds of the catalytically active components, such as the oxides, mixed oxides or hydroxides thereof, or mixtures thereof.

The elements Cu and/or Ni and/or Co are preferably used in the form of their insoluble oxides or hydroxides or mixed oxides. Particular preference is given to using copper oxides such as CuO, cobalt oxides such as CoO, nickel oxides such as NiO, mixed oxides of the general formula M¹_{z}(M²ₓO_{y}) where M¹ is an element of the alkali metals, alkaline earth metals or rare earth metals, and M² is cobalt, nickel or copper. In this formula, z=y−x. It is also possible to use mixtures thereof. Preference is given to the most thermodynamically stable polymorphs in each case.

In a particularly preferred embodiment, sparingly soluble or insoluble oxides or oxide mixtures, mixed oxides or mixtures of oxides or mixed oxides are used, which comprise both Cu and/or Co and/or Ni, and one or more elements of the alkali metals, alkaline earth metals and rare earth metals, and optionally one or more doping elements.

Particular preference is given to mixed oxides, such as the oxide mixtures which are disclosed in patent application PCT/EP2007/052013 and, before the reduction with hydrogen, comprise a) cobalt and b) one or more elements of the alkali metal group, of the alkaline earth metal group, of the group of the rare earths or zinc or mixtures thereof, elements a) and b) being present at least partly in the form of their mixed oxides, for example LiCoO₂, or

oxide mixtures, such as the oxide mixtures disclosed in EP-A-0636409, which, before the reduction with hydrogen, comprise 55 to 98% by weight of Co, calculated as CoO, 0.2 to 15% by weight of phosphorus, calculated as H₃PO₄, 0.2 to 15% by weight of manganese, calculated as MnO₂, and 0.2 to 5.0% by weight of alkali metal, calculated as M₂O (M=alkali metal), or
oxide mixtures disclosed in EP-A-0742045, which, before the reduction with hydrogen, comprise 55 to 98% by weight of Co, calculated as CoO, 0.2 to 15% by weight of phosphorus, calculated as H₃PO₄, 0.2 to 15% by weight of manganese, calculated as MnO₂, and 0.05 to 5% by weight of alkali metal, calculated as M₂O (M=alkali metal), or
oxide mixtures disclosed in EP-A-696572, which, before the reduction with hydrogen, comprise 20 to 85% by weight of ZrO₂, 1 to 30% by weight of oxygen compounds of copper, calculated as CuO, 30 to 70% by weight of oxygen compounds of nickel, calculated as NiO, 0.1 to 5% by weight of oxygen compounds of molybdenum, calculated as MoO₃, and 0 to 10% by weight of oxygen compounds of aluminum and/or manganese, calculated as Al₂O₃ and MnO₂ respectively, for example the catalyst disclosed in loc. cit., page 8, with the composition of 31.5% by weight of ZrO₂, 50% by weight of NiO, 17% by weight of CuO and 1.5% by weight of MoO₃, or
oxide mixtures disclosed in EP-A-963 975, which, before the reduction with hydrogen, comprise 22 to 40% by weight of ZrO₂, 1 to 30% by weight of oxygen compounds of copper, calculated as CuO, 15 to 50% by weight of oxygen compounds of nickel, calculated as NiO, where the molar Ni:Cu ratio is greater than 1, 15 to 50% by weight of oxygen compounds of cobalt, calculated as CoO, 0 to 10% by weight of oxygen compounds of aluminum and/or manganese, calculated as Al₂O₃ and MnO₂ respectively, and no oxygen compounds of molybdenum, for example the catalyst A disclosed in loc. cit., page 17, with the composition of 33% by weight of Zr, calculated as ZrO₂, 28% by weight of Ni, calculated as NiO, 11% by weight of Cu, calculated as CuO and 28% by weight of Co, calculated as CoO, or
copper-containing oxide mixtures disclosed in DE-A-2445303, for example the copper-containing precipitated catalyst disclosed in Example 1 there, which is prepared by treating a solution of copper nitrate and aluminum nitrate with sodium bicarbonate and subsequent washing, drying and heat treatment of the precipitate, and has a composition of approx. 53% by weight of CuO and approx. 47% by weight of Al₂O₃, or
oxide mixtures disclosed in WO 2004085356, WO 2006005505 and WO 2006005506, which comprise copper oxide (with a proportion in the range of 50≦x≦80, preferably 55≦x≦75%, by weight), aluminum oxide (with a proportion in the range of 15≦y≦35, preferably 20≦y≦30%, by weight) and lanthanum oxide (with a proportion in the range of 1≦z≦30, preferably 2 to 25, % by weight), based in each case on the total weight of the oxidic material after calcination, where: 80≦x+y+z≦100, especially 95≦x+y+z≦100, and metallic copper powder, copper flakes or cement powder or a mixture thereof with a proportion in the range from 1 to 40% by weight, based on the total weight of the oxidic material, and graphite with a proportion of 0.5 to 5% by weight, based on the total weight of the oxidic material, where the sum of the proportions of oxidic material, metallic copper powder, copper flakes or cement powder or a mixture thereof and graphite adds up to at least 95% by weight of the shaped body produced from this material.

In a very particularly preferred embodiment, the insoluble or sparingly soluble compound of the catalytically active components is LiCoO₂.

Processes for preparing LiCoO₂ are described, for example, in Antolini (E. Antolini, Solid State Ionics, 159-171 (2004)) and Fenton et al. (W. M. Fenton, P. A. Huppert, Sheet Metal Industries, 25 (1948), 2255-2259).

For instance, LiCoO₂ can be prepared by thermal treatment of the corresponding lithium and cobalt compounds, such as the nitrates, carbonates, hydroxides, oxides, acetates, citrates or oxalates.

In addition, LiCoO₂ can be obtained by precipitating water-soluble lithium and cobalt salts by adding an alkaline solution, and subsequently calcining.

LiCoO₂ can also be obtained by the sol-gel method.

LiCoO₂ can also, as described by Song et al. (S. W. Song, K. S. Han, M. Yoshimura, Y. Sata, A. Tatsuhiro, Mat. Res. Soc. Symp. Proc, 606, 205-210 (2000)), be obtained by hydrothermal treatment of cobalt metal with aqueous LiOH solutions.

In a particular embodiment, the suspension of the insoluble or sparingly soluble compounds of the catalytically active components is obtained by “precipitation”, by precipitating compounds of the catalytically active components which are soluble in the abovementioned liquid by adding a precipitant.

Useful soluble compounds of the catalytically active components generally include soluble metal salts such as the hydroxides, sulfates, carbonates, oxalates, nitrates, acetates or chlorides of the catalytically active components. The precipitation can also be effected with other suitable soluble compounds of the corresponding elements. The elements Cu and/or Co and/or Ni are preferably used in the form of their soluble carbonates, chlorides or nitrates.

The elements of the alkali metals, alkaline earth metals and rare earth metals are preferably used in the form of their soluble hydroxides, for example LiOH, KOH, NaOH, CsOH, Ca(OH)₂ or Mg(OH)₂.

Typically, the precipitation involves precipitating the soluble compounds as sparingly soluble or insoluble basic salts by adding a precipitant.

The precipitants used are preferably bases, especially mineral bases, such as alkali metal bases. Examples of precipitants are sodium carbonate, sodium hydroxide, potassium carbonate or potassium hydroxide.

The precipitants used may also be ammonium salts, for example ammonium halides, ammonium carbonate, ammonium hydroxide or ammonium carboxylates.

The precipitation can be performed, for example, at temperatures of 20 to 100° C., particularly 30 to 90° C., especially at 50 to 70° C.

The precipitates obtained in the precipitation are generally chemically inhomogeneous and generally comprise mixtures of the oxides, oxide hydrates, hydroxides, carbonates and/or hydrogencarbonates of the metals used.

In a preferred embodiment, the suspension is prepared by adding the catalytically active components in particulate form, for example as a powder, to the liquid. The embodiment has the advantage that the preparation of the suspensions is readily reproducible. In particular, the catalytically active components used in particulate form are the abovementioned preferred and particularly preferred sparingly soluble and insoluble oxides or oxide mixtures, mixed oxides or mixtures of oxides or mixed oxides which comprise both Cu and/or Co and/or Ni and one or more elements of the alkali metals, alkaline earth metals and rare earth metals, and optionally one or more doping elements.

The catalytically active components in particulate form are preferably obtained by spray drying, for example by spray drying a suspension obtained by precipitation.

The particles, present in suspension, of the insoluble or sparingly soluble compounds of the catalytically active components preferably have a mean particle diameter of 0.001 to 1000 μm, more preferably 1 to 500 μm, especially preferably of 10 to 100 μm and most preferably of 20 to 80 μm. Particles of this order of size enable a homogeneous coating and lead to catalysts which have a high activity and mechanical stability.

In order to prevent the sedimentation of the insoluble or sparingly soluble compounds of the catalytically active components in the suspension, the suspension is generally dispersed intensively, the dispersion preferably being effected by means of intensive stirring or by means of ultrasound. The dispersion can preferably also be effected by continuously pumping the suspension in circulation.

The concentration of insoluble or sparingly soluble compounds of the catalytically active components in the suspension is generally 0.1 to 50% by weight, preferably 1 to 30% by weight and more preferably 5 to 25% by weight, based in each case on the liquid used.

In particular, the concentration of the insoluble or sparingly soluble compounds of the alkali metals, alkaline earth metals and rare earth metals is 0.1 to 20% by weight, preferably 0.5 to 10% by weight and more preferably 1 to 5% by weight, based in each case on the mass of the liquid used.

The concentration of the insoluble or sparingly soluble compounds of Cu, Ni and Co is 1 to 50% by weight, preferably 5 to 25% by weight and more preferably 10 to 20% by weight, based in each case on the mass of the liquid used.

The monolithic catalyst support is coated by contacting the monolithic catalyst support with the insoluble or sparingly soluble compounds of the catalytically active components present in suspension.

Before the contacting, the monolithic catalyst support is preferably dried. The drying is effected generally at 100 to 200° C. for a duration of 1 to 48 hours.

The monolithic catalyst support is coated preferably by preparing the suspension before the contacting of the monolithic catalyst support and contacting the monolithic catalyst support with the already prepared suspension.

The monolithic catalyst support is preferably contacted with the suspension by immersing the monolithic catalyst support into the suspension or by pumping the suspension continuously over the monolithic catalyst support.

In a particularly preferred embodiment, the monolithic catalyst support is immersed into the suspension.

In a very particularly preferred embodiment, during the immersion, the suspension is sucked in through the channels of the monolithic catalyst support, such that the suspension can penetrate very substantially fully into the channels of the monolith. The suction of the suspension can be effected, for example, by generating a reduced pressure at one end of the monolithic catalyst support and immersing the other end of the monolithic catalyst support into the suspension, which sucks in the suspension.

However, the monolithic catalyst support can also be coated by virtue of the monolithic catalyst support already being suspended in the liquid and the suspension being prepared “in situ” in the liquid by “precipitation”. In this method, the insoluble or sparingly soluble compounds of the catalytically active components are precipitated directly onto the monolithic catalyst support.

The monoliths are generally contacted with the suspension by, for example, immersion until complete and homogeneous coating of the catalyst support is ensured.

The suspension is preferably dispersed during the contacting of the monolithic catalyst support, in order that the particles can penetrate very substantially fully into the channels of the monolith, and a homogeneous coating is achieved.

After the contacting, the excess of suspension is typically removed. The suspension can be removed, for example, by decanting off, dripping off, filtration or filtering off. The suspension is preferably removed by generating an elevated pressure at one end of the monolithic catalyst support and forcing the excess suspension out of the channels. The elevated pressure can, for example, be effected by blowing compressed air into the channels.

Subsequently, the coated monolithic catalyst support is generally dried and calcined. The drying is effected typically at temperatures of 80 to 200° C., preferably 100 to 150° C. The calcination is performed generally at temperatures of 300 to 800° C., preferably 400 to 600° C., more preferably 450 to 550° C.

The contacting of the monolithic catalyst support with the suspension can be repeated once or more than once.

In a particularly preferred embodiment, the inventive catalysts are prepared by a combination of impregnation and coating. Very particular preference is given to applying the elements Cu and/or Co and/or Ni to the monolithic catalyst support by coating in a first stage or a plurality of stages, and applying the elements of the alkali metals, alkaline earth metals or rare earth metals or the doping elements thereafter in one or more stages by impregnation.

This particularly preferred preparation method for the catalyst enables the application of a high proportion of the elements of the alkali metals, alkaline earth metals and rare earth metals.

In a particularly preferred embodiment, before the impregnation with the catalytically active components and before and/or during the coating of the monolithic catalyst support with the catalytically active components, a binder is applied to the monolithic catalyst support. Application of a binder to the monolithic catalyst support can increase the intrinsic surface area, thus allowing more active material to be applied, which increases the catalytic activity of the catalysts.

The binders used are preferably aluminum oxides, especially gamma- or delta-aluminum oxides, alpha-aluminum oxides, silicon dioxide, kieselguhr, titanium dioxide, zirconium dioxide, cerium dioxide, magnesium oxide, and mixtures thereof. Particularly preferred binders are aluminum oxides, especially gamma- or delta-aluminum oxides, alpha-aluminum oxides, silicon dioxide or magnesium oxide, and mixtures thereof. The binder is applied preferably by coating the monolithic catalyst support. The coating generally involves contacting the monolithic catalyst support together with a suspension (liquid which comprises binder) which comprises the binder.

The concentration of the binder in the suspension is preferably 0.5 to 25% by weight, more preferably 1 to 15% by weight and most preferably 1 to 5% by weight, based on the liquid used.

The liquids used are generally the aforementioned liquids.

In a preferred embodiment, the suspension is prepared by adding the binder in particulate form, for example as a powder, to the liquid.

The particles of the binder present in suspension preferably have a mean particle diameter of 0.001 to 1000 μm, more preferably 1 to 500 μm, especially preferably of 10 to 100 μm and most preferably of 20 to 80 μm.

In order to prevent the sedimentation of the insoluble or sparingly soluble compounds of the catalytically active components in the suspension, the suspension is generally dispersed intensively, the dispersion preferably being effected by means of intensive stirring or by means of ultrasound. The dispersion can preferably also be effected by pumping the suspension continuously in circulation.

The monolithic catalyst support is coated by contacting the monolithic catalyst support with the binder present in suspension.

The monolithic catalyst support is coated with binder preferably by preparing the suspension before the contacting of the monolithic catalyst support, and contacting the monolithic catalyst support with the already prepared suspension.

The monolithic catalyst support is preferably contacted with the suspension by immersing the monolithic catalyst support into the suspension or by pumping the suspension continuously over the monolithic catalyst support.

In a particularly preferred embodiment, the monolithic catalyst support is immersed into the suspension.

In a very particularly preferred embodiment, during the immersion, the suspension is sucked in through the channels of the monolithic catalyst support, such that the suspension can penetrate very substantially fully into the channels of the monolith. The suspension can be sucked in, for example, by generating a reduced pressure at one end of the monolithic catalyst support and immersing the other end of the monolithic catalyst support into the suspension, which sucks in the suspension.

After the contacting, the excess of suspension is removed. The suspension can be removed, for example, by decanting off, dripping off, filtration or filtering off. The suspension is preferably removed by generating an elevated pressure at one end of the monolithic catalyst support and forcing the excess suspension out of the channels. The elevated pressure can be effected, for example, by blowing compressed air into the channels.

Subsequently, the coated monolithic catalyst support is generally dried and calcined. The drying is effected typically at temperatures of 80 to 200° C., preferably 100 to 150° C. The calcination is performed generally at temperatures of 300 to 800° C., preferably 400 to 600° C., more preferably 450 to 550° C.

The contacting of the monolithic catalyst support with the suspension which comprises the binder can be repeated once or more than once.

When the catalytically active components are applied by impregnation, the monolithic catalyst support is preferably coated with binder before the impregnation.

When the catalytically active components are applied by coating, the monolithic catalyst support can be coated with binder before the coating of the catalytically active components.

In a preferred embodiment, the coating of the monolithic catalyst support with binder, however, is effected simultaneously with the coating with catalytically active components, by using a suspension which, as well as the insoluble or sparingly soluble components of the catalytically active components, additionally comprises binder in particulate form for the coating.

In a very particularly preferred embodiment, the monolithic catalyst support and/or the binder are contacted with an acid before and/or during the application of the binder. The treatment of the monolithic catalyst support and/or of the binder with acid can further increase the specific surface area of the monolith and improve the adhesion between monolithic catalyst support and binder, which enhances the mechanical stability and also the catalytic activity of the inventive catalysts.

The acids used are preferably organic acids such as formic acid or acetic acid.

The acid is preferably added directly to the suspension of binder and liquid.

The concentration of acid in the liquid is preferably 0.1 to 5% by weight, preferably 0.5 to 3% by weight, more preferably 1 to 2% by weight, based in each case on the mass of the liquid used.

The monolithic catalysts obtained by impregnation or coating generally comprise, after the calcination, the catalytically active components in the form of a mixture of oxygen compounds thereof, i.e. especially as the oxides, mixed oxides and/or hydroxides. The catalysts thus prepared can be stored as such.

Before they are used as hydrogenation catalysts, the inventive catalysts which, as described above, have been obtained by impregnation or coating are generally prereduced by treatment with hydrogen after the calcination or conditioning. They can, however, also be used in the process without prereduction, in which case they are reduced under the conditions of the hydrogenation by the hydrogen present in the reactor, which generally converts the catalyst to its catalytically active form in situ.

For prereduction, the catalysts are generally first exposed to a nitrogen-hydrogen atmosphere at 150 to 200° C. over a period of 12 to 20 hours, and then treated in a hydrogen atmosphere at 200 to 400° C. for another up to approx. 24 hours. This prereduction reduces a portion of the oxygen-containing metal compounds present in the catalysts to the corresponding metals, such that they are present in the active form of the catalyst together with the different kinds of oxygen compounds.

In a particularly preferred embodiment, the prereduction of the catalyst is undertaken in the same reactor in which the hydrogenation process according to the invention is subsequently carried out.

After the prereduction, the catalyst thus formed can be handled and stored under an inert gas such as nitrogen or under an inert liquid, for example an alcohol, water or the product of the particular reaction for which the catalyst is used. After the prereduction, the catalyst can, however, also be passivated with an oxygen-comprising gas stream such as air or a mixture of air with nitrogen, i.e. provided with a protective oxide layer. The storage of the catalysts under inert substances or the passivation of the catalyst enable uncomplicated and unhazardous handling and storage of the catalyst. It may then be necessary to free the catalyst of the inert liquid before the start of the actual reaction, or to remove the passivation layer, for example, by treatment with hydrogen or a hydrogen-comprising gas.

Before the start of the hydrogenation, the catalyst can be freed of the inert liquid or passivation layer. This is done, for example, by the treatment with hydrogen or a hydrogen-comprising gas.

Catalyst precursors can, however, also, as described above, be used in the process without prereduction, in which case they are then reduced under the conditions of the hydrogenation by the hydrogen present in the reactor, which generally forms the catalyst in situ in its active form.

The catalytic properties of the above-described catalysts can be improved by contacting the catalysts with one or more basic compounds selected from the group of the alkali metals, alkaline earth metals and rare earth metals.

Accordingly, the present invention also relates to the use of a basic compound selected from the group of the alkali metals, alkaline earth metals and rare earth metals for improving the activity of a catalyst, especially of hydrogenation catalysts comprising copper and/or cobalt and/or nickel, said catalyst being present in the form of a structured monolith.

The improvement in the catalytic properties may consist, for example, in increasing the selectivity and/or the activity of the catalysts. However, an improvement in the catalytic properties may also mean that the service life of the above-described catalysts is increased and the catalyst over a longer period the catalytic activity and/or selectivity of the catalyst is maintained without significant losses. An improvement in the catalytic properties may additionally mean that the catalytic properties which have declined, for example, in the course of a long operating time, are reestablished (regeneration of the catalyst).

In a particularly preferred embodiment, the above-described catalysts are contacted by contacting the basic compound with the catalyst as a solution before, after or during use of the catalyst in a reaction.

A reaction is understood to mean the conversion of one or more reactants over the above-described catalysts.

The catalyst can be contacted with a basic compound selected from the group of the alkali metals, alkaline earth metals and rare earth metals before the catalyst is used in the reaction, for example, by contacting the catalyst as described above, in the course of its preparation, with a basic compound selected from the group of the alkali metals, alkaline earth metals and rare earth metals, for example by impregnating a monolithic catalyst support which has preferably been coated with Ni, Co and/or Cu with one or more soluble compounds of the alkali metals, alkaline earth metals and rare earth metals.

In a particularly preferred embodiment, the catalysts are contacted for the first time and/or additionally during a reaction with one or more soluble compounds of the elements selected from the group of the alkali metals, alkaline earth metals and rare earth metals.

The catalyst is contacted during a reaction more preferably by introducing a solution of the basic compound into the reactor together with the reactant stream and/or adding it together with the reactants. Particular preference is given to adding solutions of the basic compounds in water or other suitable solvents, such as alkanols, such as C1-C4-alkanols, e.g. methanol or ethanol, or ethers, such as cyclic ethers, e.g. THF or dioxane, to the reaction mixture. Particular preference is given to adding solutions of alkali metal or alkaline earth metal hydroxides or of hydroxides of the rare earth metals in water, more preferably solutions of LiOH, NaOH, KOH and/or CsOH in water. The concentration of the basic compound in water or other suitable solvents is preferably 0.01 to 20% by weight, preferably 0.1 to 10% by weight and more preferably 0.2 to 5% by weight.

The amount of the solution of the basic compound added is typically selected such that the ratio of the mass of the basic compound added to the mass of the reactants to be converted in the reaction mixture is 100 to 10 000:1 000 000, preferably 150 to 5000:1 000 000 and more preferably 200 to 1000:1 000 000.

The feeding can be effected over the entire reaction time or only during part of the entire reaction time.

The solution of the basic compounds is preferably fed in over the entire duration of the reaction.

An improvement in the catalytic properties can also be achieved by contacting a catalyst after a reaction with a solution of a basic compound selected from the group of the alkali metals, alkaline earth metals and rare earth metals. The contacting can be effected, for example, by impregnating the catalyst, after the reaction, with a solution of a basic compound or passing a solution of a basic compound over the catalyst. The contacting of the catalyst after the reaction can bring about at least a partial regeneration of the catalytic properties.

The contacting of the basic compound can, as mentioned above, be effected, for example, in the course of or during the preparation, for example by coating the catalyst in the presence of a basic compound, by impregnating the monolithic catalyst support with a basic compound and/or impregnating a coated monolithic catalyst support with a basic compound.

When the contacting follows a calcination, the compounds of Cu, Ni and Co are generally present in the form of their oxidic compounds, for example as oxides, mixed oxides and/or hydroxides.

In a preferred embodiment, the catalyst is contacted with the basic compound after the catalyst has been reduced and is present in reduced form. The catalyst is more preferably contacted with the basic compound in the presence of hydrogen. The contacting is most preferably effected during a hydrogenation reaction in the presence of hydrogen.

The process according to the invention allows the catalytic properties of the aforementioned catalysts to be improved especially when the reaction in which the catalyst is used or is to be used is a process for hydrogenating compounds which comprise at least one unsaturated carbon-carbon, carbon-nitrogen or carbon-oxygen bond.

Suitable compounds are generally compounds which comprise at least one or more than one carboxamide group, nitrile group, imine group, enamine group, azine group or oxime group, which are hydrogenated to amines.

In addition, it is possible in the process according to the invention to hydrogenate compounds which comprise at least one or more than one carboxylic ester group, carboxylic acid group, aldehyde group or keto group to alcohols.

Suitable compounds are also aromatics, which can be converted to unsaturated or saturated carbo- or heterocycles.

Particularly suitable compounds which can be used in the process according to the invention are organic nitrile compounds, imines and organic oximes. These can be hydrogenated to primary amines.

In a very particularly preferred embodiment, nitriles are used in the process according to the invention.

The hydrogenation may, for example, be that of aliphatic mono- and dinitriles having 1 to 30 carbon atoms, of cycloaliphatic mono- and dinitriles having 6 to 20 carbon atoms, or else that of alpha- and beta-amino nitriles or alkoxynitriles.

Suitable nitriles are, for example, acetonitrile to prepare ethylamine, propionitrile to prepare propylamine, butyronitrile to prepare butylamine, lauronitrile to prepare laurylamine, stearylnitrile to prepare stearylamine, N,N-dimethylaminopropionitrile (DMAPN) to prepare N,N-dimethylaminopropylamine (DMAPA) and benzonitrile to prepare benzylamine. Suitable dinitriles are adiponitrile (ADN) to prepare hexamethylenediamine (HMD) or HMD and 6-aminocapronitrile (ACN), 2-methyl-glutaronitrile to prepare 2-methylglutaramine, succinonitrile to prepare 1,4-butane-diamine and suberonitrile to prepare octamethylenediamine. Also suitable are cyclic nitriles such as isophoronenitrile imine (isophoronenitrile) to prepare isophoronediamine, and isophthalonitrile to prepare meta-xylylenediamine. Equally suitable are α-amino nitriles and β-amino nitriles, such as aminopropionitrile to prepare 1,3-diaminopropane, or ω-amino nitriles, such as aminocapronitrile to prepare hexamethylenediamine. Further suitable compounds are so-called “Strecker nitriles”, such as iminodiacetonitrile to prepare diethylenetriamine. Further suitable nitriles are β-amino nitriles, for example addition products of alkylamines, alkyldiamines or alkanolamines onto acrylonitrile. For instance, it is possible to convert addition products of ethylenediamine and acrylonitrile to the corresponding diamines. For example, 3-[2-aminoethyl]aminopropionitrile can be converted to 3-(3-aminoethyl)aminopropylamine, and 3,3′-(ethylenediimino)bispropionitrile or 3-[2-(aminopropylamino)ethylamino]-propionitrile to N,N′-bis(3-aminopropyl)ethylenediamine.

Particular preference is given to using N,N-dimethylaminopropionitrile (DMAPN) to prepare N,N-dimethylaminopropylamine (DMAPA), adiponitrile (ADN) to prepare hexamethylenediamine (HMD) or 6-aminocapronitrile (6-ACN) and HMD, and isophoronenitrile imine to prepare isophoronediamine in the process according to the invention.

The reducing agents used may be hydrogen or a hydrogen-comprising gas. The hydrogen is generally used in technical grade purity. The hydrogen can also be used in the form of a hydrogen-comprising gas, i.e. in mixtures with other inert gases, such as nitrogen, helium, neon, argon or carbon dioxide. The hydrogen-comprising gases used may, for example, be reformer offgases, refinery gases, etc., if and when these gases do not comprise any catalyst poisons for the hydrogenation catalysts used, for example CO. Preference is given, however, to using pure hydrogen or essentially pure hydrogen in the process, for example hydrogen with a content of more than 99% by weight of hydrogen, preferably more than 99.9% by weight of hydrogen, more preferably more than 99.99% by weight of hydrogen, especially more than 99.999% by weight of hydrogen.

The molar ratio of hydrogen to the compound used as the reactant is generally 1:1 to 25:1, preferably 2:1 to 10:1. The hydrogen can be recycled into the reaction as cycle gas.

In a process for preparing amines by reducing nitriles, the hydrogenation can be effected with addition of ammonia. In this case, ammonia is generally used in molar ratios relative to the nitrile group in a ratio of 0.5:1 to 100:1, preferably 2:1 to 20:1. However, the preferred embodiment is a process in which no ammonia is added.

The reaction can be performed in bulk or in a liquid.

The hydrogenation is effected preferably in the presence of a liquid.

Suitable liquids are, for example, C1- to C4-alcohols, such as methanol or ethanol, C4- to C12-dialkyl ethers, such as diethyl ether or tert-butyl methyl ether, or cyclic C4- to C12-ethers, such as tetrahydrofuran or dioxane. Suitable liquids may also be mixtures of the aforementioned liquids. The liquid may also be the product of the hydrogenation. The reaction can also be effected in the presence of water. The water content, however, should not be more than 10% by weight, preferably less than 5% by weight, more preferably less than 3% by weight, based on the mass of the liquid used, in order to very substantially prevent the compounds of the alkali metals, alkaline earth metals and/or rare earth metals from being leached out and/or washed off.

The hydrogenation is performed generally at a pressure of 1 to 150 bar, especially of 5 to 120 bar, preferably of 8 to 85 bar and more preferably of 10 to 65 bar. Preference is given to performing the hydrogenation at a pressure of less than 65 bar as a low-pressure process. The temperature is generally within a range of 25 to 300° C., especially from 50 to 200° C., preferably from 70 to 150° C., more preferably from 80 to 130° C.

The hydrogenation process according to the invention can be performed continuously, batchwise or semicontinuously. Preference is given to hydrogenating semicontinuously or continuously.

Suitable reactors are thus both stirred tank reactors and tubular reactors. Typical reactors are, for example, high-pressure stirred tank reactors, autoclaves, fixed bed reactors, fluidized bed reactors, moving beds, circulating fluidized beds, continuous stirred tanks, bubble reactors, circulation reactors, for example jet loop reactors, etc., the reactor suitable for the desired reaction conditions (such as temperature, pressure and residence time) being used in each case.

The reactors may each be used as a single reactor, as a series of single reactors and/or in the form of two or more parallel reactors.

The reactors can be operated in an AB mode (alternating mode). The process according to the invention can be performed as a batchwise reaction, semicontinuous reaction or continuous reaction.

The specific reactor construction and the performance of the reaction may vary depending on the hydrogenation process to be performed, the state of matter of the starting material to be hydrogenated, the reaction times required and the nature of the catalyst used.

In a very particularly preferred embodiment, the process according to the invention for hydrogenation is performed continuously in a high-pressure stirred tank reactor, a bubble column, a circulation reactor, for instance a jet loop reactor, or a fixed bed reactor in which the catalyst is arranged in a fixed manner, i.e. in the form of a fixed catalyst bed. It is possible to hydrogenate in liquid phase mode or trickle mode, preferably in liquid phase mode. Working in liquid phase mode is found to be technically simpler.

In this preferred embodiment, the advantages of the inventive catalysts are shown particularly efficiently, since the inventive catalysts have a high mechanical stability and hence high service lives, which makes them suitable for continuous processes. In a particularly preferred embodiment, the hydrogenation of nitriles is performed continuously in the liquid phase with a catalyst arranged in a fixed manner in a stirred autoclave, a bubble column, a circulation reactor, for instance a jet loop, or a fixed bed reactor.

The catalyst hourly space velocity in continuous mode is typically 0.01 to 10, preferably 0.2 to 7 and more preferably 0.5 to 5 kg of reactant per l of catalyst and hour.

In a preferred embodiment, the contacting of the catalyst during the continuous hydrogenation in the liquid phase is effected, as described above, by introducing a solution of a basic compound of one or more elements selected from the group of the alkali metals, alkaline earth metals and rare earth metals together with the reactants to be hydrogenated.

Since, as described above, the reaction is preferably effected under a high pressure, it is therefore generally necessary to undertake a metered addition of the solution of a basic compound at a high operating pressure in the reactor. Suitable industrial apparatus for metered addition of substances under high pressure conditions is known to those skilled in the art. More particularly, it is possible to use pumps such as high-pressure pumps and piston pumps for metered addition of substances under high pressure conditions.

In the case of batchwise hydrogenation in the liquid phase, a suspension of reactant and catalyst is generally initially charged in the reactor. In order to ensure a high conversion and high selectivity, the suspension of reactant and catalyst has to be mixed thoroughly with hydrogen, for example by means of a turbine stirrer in an autoclave. The suspended catalyst material can be introduced and removed again with the aid of customary techniques (sedimentation, centrifugation, cake filtration, crossflow filtration). The catalyst can be used once or more than once. The catalyst concentration is advantageously 0.1 to 50% by weight, preferably 0.5 to 40% by weight, more preferably 1 to 30% by weight, especially 5 to 20% by weight, based in each case on the total weight of the suspension consisting of reactant and catalyst. The reactants can optionally be diluted with a suitable inert solvent.

The residence time in the process according to the invention in the case of performance in a batchwise process is generally 15 minutes to 72 hours, preferably 60 minutes to 24 hours, more preferably 2 hours to 10 hours.

In a particularly preferred embodiment, the contacting of the catalyst during the batchwise hydrogenation is effected by metered addition of a solution of a basic compound of one or more elements selected from the group of the alkali metals, alkaline earth metals and rare earth metals together with the reactants to be hydrogenated. The solution of the basic compound is generally initially charged together with the reactants, such that the basic compound is in contact with the catalyst over the entire reaction time.

However, the contacting can also be effected by adding the basic compound before the reaction, separately or together with the reactants. The basic compound can also be added in solid form when it is at least partially soluble in the reaction medium.

The hydrogenation can likewise be performed in the gas phase in a fixed bed reactor or a fluidized bed reactor. Common reactors for performing hydrogenation reactions are described, for example, in Ullmann's Encyclopedia [Ullmann's Encyclopedia Electronic Release 2000, chapter: Hydrogenation and Dehydrogenation, p. 2-3].

The contacting of the catalyst in the course of hydrogenation in the gas phase is preferably effected by applying the catalyst to the catalyst by impregnation before the reaction with a basic compound of one or more elements selected from the group of the alkali metals, alkaline earth metals and rare earth metals.

The activity and/or selectivity of the inventive catalysts can decrease with increasing service life. Accordingly, a process has been found for regenerating the inventive catalysts, in which the catalyst is treated with a liquid. The treatment of the catalyst with a liquid should lead to any adhering compounds which block active sites of the catalyst being detached. The treatment of the catalyst with a liquid can be effected by stirring the catalyst in a liquid or by washing the catalyst in the liquid, and, on completion of treatment, the liquid can be removed from the catalyst together with the detached impurities by filtering or decanting off.

Suitable liquids are generally the product of the hydrogenation, water or an organic solvent, preferably ethers, alcohols or amides.

In a further embodiment, the catalyst can be treated with liquid in the presence of hydrogen or of a hydrogen-comprising gas.

This regeneration can be performed under elevated temperature, generally of 20 to 250° C. It is also possible to dry the spent catalyst and to oxidize adhering organic compounds with air to volatile compounds such as CO₂. Before a further use of the catalyst in the hydrogenation, on completion of oxidation, it generally has to be activated as described above.

In the regeneration, the catalyst can be contacted with a soluble compound of the catalytically active components. The contacting can be effected in such a way that the catalyst is impregnated or wetted with a water-soluble compound of the catalytically active components. More particularly, the compound of the catalytically active components is a compound of a doping element or a compound of the metals of the alkali metals, alkaline earth metals or rare earth metals.

More preferably, the catalyst is contacted after the regeneration with a basic compound of one or more elements selected from the group of the alkali metals, alkaline earth metals and rare earth metals, preferably by impregnating the catalyst, as described above, with one or more elements selected from the group of the alkali metals, alkaline earth metals and rare earth metals, or by metering a basic compound in during the subsequent reaction.

One advantage of the invention is that the catalytic properties of catalysts present in the form of a structured monolith are improved.

In particular, the formation of undesired by-products, more particularly the formation of secondary amines from nitriles, is reduced, such that the target products are obtained in a high yield and selectivity. Moreover, the service life of the catalysts is improved and losses of selectivity and activity are reduced with increasing operating time. The process according to the invention can additionally be utilized to reestablish the catalytic properties of spent catalysts (regeneration).

The invention is illustrated by the following examples:

### DEFINITIONS

The catalyst hourly space velocity is reported as the quotient of the amount of reactant in the feed and the product of catalyst volume and time.

Catalyst hourly space velocity=amount of reactant/(volume of catalyst·reaction time). The catalyst volume corresponds to the volume that would be occupied by a solid cylinder having an outer geometry identical to the catalyst (monolith).

The reactor is generally completely filled with the monolithic catalyst.

The unit of catalyst hourly space velocity is reported in [kg_{reactant}/(l·h)].

The selectivities reported were determined by gas chromatography analyses and calculated from the area percentages.

The reactant conversion C(R) is calculated by the following formula:

$$C ( R ) = \frac{A \% {( R )}_{start} - A \% {( R )}_{end}}{A \% {( R )}_{start}}$$

The yield of product Y(P) is calculated from the area percentages of the product signal.

Y(P)=A%(P),

where the area percentages A % (i) of a reactant (A % (R)), of a product (A % (P)), of a by-product (A % (B)) or quite generally of a substance i (A % (i)), are calculated from the quotient of the area A(i) below the signal of the substance i and the total area Aₜₒₜₐₗ, i.e. the sum of the areas below the signals i, multiplied by 100:

$$A \% ( i ) = \frac{A ( i )}{{A}_{total}} \cdot 100 = \frac{A ( i )}{{\sum }_{i} A ( i )} \cdot 100$$

The selectivity of the reactant S(R) is calculated as the quotient of product yield Y(P) and reactant conversion C(R):

$$S ( R ) = \frac{Y ( P )}{C ( R )} * 100$$

The metal contents reported in the examples were obtained by elemental analysis of the finished catalyst precursors and should be interpreted as percent by weight of metal based on the total mass of the finished coated monolith (=catalyst precursor).

The examples adduced here were carried out with cordierite monoliths (Celcor®) from Corning, but can likewise be obtained with comparable monoliths (e.g. HoneyCeram® from NGK Insulators).

### EXAMPLE 1

The monolithic catalyst support was coated with an oxide mixture to EP-B1-636409. According to the method specified there, the oxide mixture may comprise 55 to 98% by weight of cobalt, 0.2 to 15% by weight of phosphorus, 0.2 to 15% by weight of manganese and 0.2 to 5% by weight of alkali metal (calculated as the oxide). The exact composition of the oxide mixture used is stated in the respective examples.

### EXAMPLE 1a

The monolithic catalyst supports used were cordierite monoliths (Celcor®) from Corning in the form of structured shaped bodies (round, 20×50 mm) and 400 cpsi.

The monolithic catalyst support was dried at 120° C. for 10 hours.

In an initial charge, 9 g of gamma-aluminum oxide (Pural SB from Sasol) were surface etched with 3 g of formic acid.

Thereafter, 300 g of an oxide mixture comprising 92% by weight of Co₃O₄, and also 5% by weight of Mn₃O₄ and 3% by weight of sodium phosphate in the 20 to 50 μl particle size fraction, which was obtained by spray drying, were added to this mixture.

300 g of demineralized water were added to this mixture and the resulting suspension was homogenized with a high-performance disperser (Ultra-Turrax from IKA).

The dry monolith was immersed into the suspension, blown dry with compressed air and dried on a hot air blower at approx. 140° C. These steps were repeated for a total of 6 immersions. Subsequently, the monolith was calcined at 500° C. for 3 hours. The catalyst precursor had a mean cobalt content of 26.1% by weight (reported as metallic cobalt).

The molar ratio of cobalt atoms to sodium atoms in the catalyst was 125:1.

### EXAMPLE 1b

The monolithic catalyst supports used were cordierite monoliths (Celcor®) from Corning in the form of structured shaped bodies (round, 18×50 mm) and 900 cpsi.

The monolithic catalyst support was dried at 120° C. for 10 hours.

In an initial charge, 7 g of gamma-aluminum oxide (Pural SB from Sasol) were surface etched with 2 g of formic acid.

Thereafter, 225 g of an oxide mixture comprising 92% by weight of CO₃O₄, and also 5% by weight of Mn₃O₄ and 3% by weight of sodium phosphate in the 20 to 50 μm particle size fraction, which was obtained by spray drying, were added to this mixture.

Approx. 400 g of demineralized water were added to this mixture and the resulting suspension was homogenized with a high-performance disperser (Ultra-Turrax from IKA).

The dry monolith was immersed into the suspension, blown dry with compressed air and dried on a hot air blower at approx. 140° C. (±10° C.). These steps were repeated for a total of 6 immersions. Subsequently, the monolith was calcined at 500° C. for 3 hours. The catalyst precursor obtained had a mean cobalt content of 14.5% by weight (reported as metallic cobalt).

The molar ratio of cobalt atoms to sodium atoms in the catalyst was 125:1.

### EXAMPLE 2

The monolithic catalyst supports used were cordierite monoliths (Celcor®) from Corning in the form of structured shaped bodies (round, 18×50 mm) and 900 cpsi.

The monolithic catalyst support was dried at 120° C. for 10 hours.

In an initial charge, 9 g of gamma-aluminum oxide (Pural SB from Sasol) were surface etched with 3 g of formic acid. Thereafter, 310 g of LiCoO₂ (Alfa Aesar: 97%) were added to this mixture which was supplemented with approx. 200 g of demineralized water, and the resulting suspension was homogenized with a high-performance disperser (Ultra-Turrax from IKA).

The dry monolith was immersed into the suspension, blown dry with compressed air and dried on a hot air blower at approx. 140° C. (±10° C.). These steps were repeated for a total of 6 immersions. Subsequently, the monolith was calcined at 500° C. for 3 hours. The catalyst precursor had a mean cobalt content of 30.5% by weight (reported as metallic cobalt) and a lithium content of 3.7% by weight (reported as metallic lithium).

The molar ratio of cobalt atoms to lithium atoms in the catalyst was 1:1.

### EXAMPLE 3

A cobalt hexaamine solution was prepared by dissolving 634 g of ammonium carbonate in 1709 ml of ammonia solution (33% NH₃). Subsequently, 528 g of cobalt(II) carbonate hydrate were added in portions. The solution was filtered to remove insoluble constituents. The resulting solution had a Redox potential of −248 mV; the cobalt content was 4% by weight.

The monolithic catalyst support used was cordierite monoliths (Celcor®) from Corning in the form of structured shaped bodies (round, 9.5×20 mm) and 400 cpsi.

The monolithic catalyst support was dried at 120° C. for 10 hours.

In an initial charge, 7.9 g of gamma-aluminum oxide (Pural SB from Sasol) were surface etched with 2.4 g of formic acid. 256 g of gamma-aluminum oxide (D10-10, BASF SE) were mixed with the surface etched gamma-aluminum oxide and added to the cobalt hexaamine solution.

The dry monolith was immersed into the suspension thus prepared, blown dry with compressed air and dried on a hot air blower at approx. 140° C. (±10° C.). These steps were repeated for a total of 4 immersions. Subsequently, the monolith was dried at 105° C. in a drying cabinet for 2 hours and calcined at 280° C. for 4 hours. The catalyst precursor had a mean cobalt content of 1.0% by weight (reported as metallic cobalt).

### EXAMPLE 4

The monolithic catalyst support used was cordierite monoliths (Celcor®) from Corning in the form of structured shaped bodies (round, 9.5×20 mm) and 400 cpsi.

The monolithic catalyst support was dried at 120° C. for 10 hours.

In an initial charge, 2.1 g of aluminum oxide (Disperal, SOL 73, ground) was surface etched with 0.6 g of glacial acetic acid (100%).

Thereafter, 65.5 g of an oxide mixture comprising 71% by weight of NiO, and also 20.4% by weight of Al₂O₃, 8.5% by weight of ZrO₂ and 0.04% by weight of Na₂O in the 20 to 50 μm particle size fraction, which had been obtained by spray drying, were added to this mixture.

Approx. 160 g of demineralized water were added to this mixture and the resulting suspension was homogenized with a high-performance disperser (Ultra-Turrax from IKA).

The dry monolith was immersed into the suspension, blown dry with compressed air and dried on a hot air blower at approx. 140° C. (±10° C.). These steps were repeated for a total of 5 immersions. Subsequently, the monolith was dried at 120° C. for 10 hours and calcined at 350° C. for 2 hours. The resulting catalyst precursor had a mean nickel content of 8.6% by weight (reported as metallic nickel).

The molar ratio of cobalt atoms to sodium atoms in the catalyst was 730:1.

### EXAMPLE 5

A catalyst precursor prepared according to Example 1a was reduced at 300° C. with a mixture of 90% hydrogen and 10% nitrogen for 10 hours, and then passivated with air at room temperature. The passivated monolith extrudates were subsequently installed into 11 bores provided in a holder, such that the bores were filled completely by the monolith extrudates.

To activate the passivated catalyst, the holder with the monoliths was installed into a 160 ml Parr autoclave (from hte) with a magnetically coupled disk stirrer (stirrer speed 1000 revolutions/minute), electrical heating, internal thermometer and hydrogen supply via iterative differential pressure metering.

The passivated catalyst was activated before the nitrile hydrogenation at 150° C./100 bar over a period of 12 hours with hydrogen while the monolithic catalysts were stirred in THF.

The holder with the activated cobalt monolith catalysts (13% by weight of cobalt) was deinstalled from the autoclave and rinsed off with THF. In Example 5a, the holder was installed into the reactor without further treatment. Alternatively, the holder was stored at room temperature for 30 minutes in an aqueous 0.85 molar solution of the alkali metal hydroxides LiOH, NaOH, KOH or CsOH (Examples 5b to 5e), which completely wetted the monolithic catalysts with the solution (impregnation).

To perform the semibatchwise hydrogenations of 3-dimethylaminopropionitrile (DMAPN) to 3-dimethylaminopropylamine (DMAPA), the autoclave was charged with 18.0 g of 3-dimethylaminopropionitrile (DMAPN), 18.0 g of THF and 25.1 g of 3-dimethylaminopropylamine. The holder with the activated, optionally base-impregnated catalysts was installed into the filled autoclave. The hydrogenation was performed under inert gas (nitrogen) at 100° C. and 100 bar for 1.5 hours. After this time, the composition of the reaction mixture was analyzed by gas chromatography.

The amount of the initially charged 3-dimethylaminopropylamine was deducted when calculating the conversion and the selectivity (Table 1).

TABLE 1

| | Impregnation | DMAPN | DMAPA |
|---|---|---|---|
| Experiment No. | with bases | conversion [%] | selectivity [%] |
| 5a | — | 99.2 | 83.3 |
| 5b | LiOH | 99.2 | 97.0 |
| 5c | NaOH | 99.7 | 95.4 |
| 5d | KOH | 99.9 | 96.4 |
| 5e | CsOH | 99.8 | 95.0 |

### EXAMPLE 6

The hydrogenation was performed in a bubble column which comprised a catalyst according to Example 1a, 1b or a catalyst prepared according to Example 2, in stacked form, in liquid phase mode. The hydrogenation effluent was separated into gas and liquid phase in a phase separation vessel. The liquid phase was discharged and analyzed quantitatively by GC analysis. 99.2 to 99.9% of the liquid phase was recycled into the bubble column together with the fresh DMAPN and the fresh hydrogen.

### EXAMPLE 6a

Catalyst prepared according to Example 1a (11 monoliths 20.4×50 mm, 1 monolith 20.4×18.5 mm) was reduced with hydrogen at 120° C. and 60 bar in THF for 18 hours. The THF was discharged and the apparatus (bubble column+catalyst) was then purged at room temperature with 800 ml of a 2% by weight aqueous LiOH solution for 60 minutes. Subsequently, the aqueous solution was discharged and the system was purged twice with 800 ml of tetrahydrofuran each time for 10 minutes. DMAPN was then conducted continuously into the THF-filled reactor.

The hydrogenation of 3-dimethylaminopropionitrile (DMAPN) to 3-dimethylamino-propylamine (DMAPA) was conducted in liquid phase mode in the absence of ammonia at 120° C., a pressure range of 30 to 50 bar and a WHSV of 0.26 kg/l·h of DMAPN to 0.4 kg/l.·h of DMAPN for 500 hours. The DMAPN conversion was complete; the DMAPA yield was 99.0% to 99.7%. The proportion of bis-DMAPA was accordingly less than 1%.

### EXAMPLE 6b

Catalyst precursors prepared according to Example 1b were reduced as in Example 6a, treated with lithium hydroxide solution and then rinsed with tetrahydrofuran. The hydrogenation of DMAPN was effected in the apparatus described in Example 6a. It was conducted in the absence of ammonia at 120° C. in liquid phase mode, a pressure range of 30 to 50 bar and a WHSV of 0.26 kg/l·h of DMAPN for 300 hours. The DMAPN conversion was complete; the DMAPA yield was >99.8%.

### EXAMPLE 6c

The passivated catalyst precursor prepared according to Example 2 proceeding from cordierite, gamma-aluminum oxide and LiCoO₂ was activated with hydrogen in the bubble column at 130° C. and 50 bar for 18 hours. Then, without washing or other aftertreatments of the monolith, DMAPN was pumped continuously into the reactor at 120° C. and 50 bar in liquid phase mode in the absence of ammonia. The WHSV was 0.26 kg/l·h of DMAPN. These conditions were maintained for 75 hours. Within this time, the conversion was complete; the yield was 99.9%. These values also remained constant for the next 50 hours after the pressure had been lowered to 30 bar. In the next 200 hours, under otherwise constant conditions, the WHSV was increased stepwise from 0.26 kg/l·h of DMAPN to 1.04 kg/l·h of DMAPN. The only change was that the conversion declined to 99.7%; the selectivity was 99.9%. For the next 115 hours, the temperature was increased to 130° C. at a WHSV of 1.1 kg/l·h of DMAPN. The conversion was then 99.8%, and the selectivity was the same.

### EXAMPLE 7

For the hydrogenation of suberonitrile to octamethylenediamine, an LiCoO₂-coated monolith catalyst prepared analogously to Example 2 was used. The monolithic catalyst support used was cordierite from Corning in the form of structured shaped bodies (round, 18×50 mm) and 400 cpsi.

The cobalt content of the monolith extrudates was 24 to 29% by weight, the lithium content 2 to 4% by weight.

The catalyst precursor was reduced at 300° C. with a mixture of 90% hydrogen and 10% nitrogen for 10 hours, and then passivated with air at room temperature. The passivated monolith extrudates were subsequently installed into 11 bores provided in a holder, such that the bores were filled completely by the monolith extrudates.

To activate the passivated catalyst, the holder with the monoliths was installed into a 160 ml Parr autoclave (from hte) with a magnetically coupled disk stirrer (stirrer speed 1000 revolutions/minute), electrical heating, internal thermometer and hydrogen supply via iterative differential pressure metering.

The passivated catalyst was activated before the nitrile hydrogenation at 150° C./100 bar over 12 hours with hydrogen while the monolithic catalysts were stirred in THF.

11 monolith catalyst extrudates were installed into the autoclave, and 43 g of suberonitrile and 43 g of methanol were introduced. Hydrogenation was effected at 100° C. and 65 bar for 3 hours. The gas chromatography analysis of the hydrogenation effluent showed an octamethylenediamine selectivity of 95.9% at a suberonitrile conversion of 99.4%.

### EXAMPLE 8

A catalyst precursor prepared according to Example 3 was reduced at 300° C. with a mixture of 90% hydrogen and 10% nitrogen for 10 hours, and then passivated with air at room temperature. The passivated monolith extrudates were subsequently installed into 11 bores provided in a holder, such that the bores were filled completely by the monolith extrudates.

To activate the passivated catalyst, the holder with the monoliths was installed into a 160 ml Parr autoclave (from hte) with a magnetically coupled disk stirrer (stirrer speed 1000 revolutions/minute), electrical heating, internal thermometer and hydrogen supply via iterative differential pressure metering.

The passivated catalyst was activated before the nitrile hydrogenation at 150° C./100 bar over 12 hours with hydrogen while the monolithic catalysts were stirred in THF.

The holder with the activated cobalt monolith catalysts (1% by weight of cobalt) was deinstalled from the autoclave and rinsed off with THF. The holder was subsequently either installed into the reactor without further treatment (Example 8a) or stored at room temperature for 30 minutes in an aqueous 0.065 molar or 0.85 molar solution of the alkali metal hydroxide LiOH (Example 8b and Example 8c respectively), which completely wetted the monolithic catalysts with the solution (impregnation).

To perform the semibatchwise hydrogenations of 3-dimethylaminopropionitrile

(DMAPN) to 3-dimethylaminopropylamine (DMAPA), the autoclave was charged with 18.0 g of 3-dimethylaminopropionitrile (DMAPN), 18.0 g of THF and 25.1 g of 3-dimethylaminopropylamine. The holder with the activated, optionally base-impregnated catalysts was installed into the filled autoclave. The hydrogenation was performed under inert gas (nitrogen) at 100° C. and 100 bar for 6 hours. After this time, the composition of the reaction mixture was analyzed by gas chromatography. The amount of the initially charged 3-dimethylaminopropylamine was deducted when calculating the conversion and the selectivity (table 2).

TABLE 2

| | Impregnation with | DMAPN | DMAPA |
|---|---|---|---|
| Experiment No. | bases | conversion [%] | selectivity [%] |
| 8a | — | 33.8 | 85.8 |
| 8b | LiOH (0.065 molar) | 49.4 | 83.2 |
| 8c | LiOH (0.85 molar) | 50.7 | 83.7 |

### EXAMPLE 9

Analogously to Example 5, an NiO-coated monolith catalyst produced according to Example 4 was used for the conversion of DMAPN to DMAPA under otherwise unchanged reaction conditions. In a departure from Example 5, the reaction was performed for 6 h.

The holder with the activated nickel monolith catalysts (8.6% by weight of nickel) was deinstalled from the autoclave and rinsed off with THF. The holder was subsequently either installed into the reactor without further treatment (Example 9a) or stored at room temperature for 30 minutes in an aqueous 0.85 molar solution of the alkali metal hydroxide LiOH (Example 9b), which completely wetted the monolithic catalysts with the solution (impregnation).

The results are shown in table 3.

TABLE 3

| Experiment | Impregnation with | DMAPN | DMAPA |
|---|---|---|---|
| No. | bases | conversion [%] | selectivity [%] |
| 9a | — | 96.6 | 50.9 |
| 9b | LiOH | 97.4 | 90.8 |

1.-15. (canceled)

16. A process for hydrogenating compounds comprising at least one unsaturated carbon-carbon, carbon-nitrogen or carbon-oxygen bond in the presence of a catalyst comprising one or more elements selected from the group consisting of cobalt, nickel and copper, said catalyst being present in the form of a structured monolith, the process comprising:
a) impregnating or coating the structured monolith with Co, Ni and/or Cu,
b) drying and calcining the impregnated or coated structured monolith; and
c) reducing the calcined structured monolith obtainable in step b);
contacting the structured monolith with a soluble basic compound after step b) or after step c) or during hydrogenation.

17. The process according to claim 16, wherein the original compound comprises at least one nitrile group and the resulting compound is a primary amine.

18. The process according to claim 17, wherein the resulting primary amine is hexamethylenediamine, aminocapronitrile, N,N-dimethylaminopropylamine or isophoronediamine.

19. The process according to claim 17, wherein the process is performed in the absence of ammonia.

20. The process according to claim 16, wherein the catalyst is contacted before the hydrogenation with one or more basic compounds selected from the group of the alkali metals, alkaline earth metals and rare earth metals.

21. The process according to claim 16, wherein the catalyst is contacted during the hydrogenation with one or more basic compounds selected from the group of the alkali metals, alkaline earth metals and rare earth metals.

22. The process according to claim 16, wherein the basicity of the reaction mixture is increased by adding a basic compound as a solution.

23. The process according to claim 22, wherein the ratio of the mass of the basic compound added to the mass of the reactant to be hydrogenated in the reactant stream is 100 to 10,000:1,000,000.

24. The process according to claim 16, wherein the hydrogenation is performed continuously and the structured monolith is arranged as a fixed bed.
